(19) United States
(12) Patent Application Publication
(10) Pub. No.: US 2025/0369033 A1
(43) Pub. Date: Dec. 4, 2025

(54) APPARATUS FOR MEASURING ENZYME ACTIVITY USING SUBSTRATE-IMMOBILIZED CARRIER WITH NON-UNIFORM SUBSTRATE IMMOBILIZATION DENSITY AND METHOD THEREOF

(71) Applicant: CANON MEDICAL SYSTEMS CORPORATION, Otawara-shi (JP)

(72) Inventor: Hiroto SATAKE, Boston, MA (US)

(73) Assignee: CANON MEDICAL SYSTEMS CORPORATION, Otawara-shi (JP)

(21) Appl. No.: 19/223,613

(22) Filed: May 30, 2025

(30) Foreign Application Priority Data
May 31, 2024 (JP) 2024-089553

(51) Int. Cl. C12Q 1/48

(52) U.S. Cl. CPC C12Q 1/48; C12Q 1/485; G01N 2333/91177; G01N 2333/91205

(57) ABSTRACT

A simpler method for measuring enzyme activity is provided. The-method for estimating enzyme activity uses a base on which spots of immobilized substrates are formed, and includes (a) enabling an enzyme to act on the substrate on the spot to obtain an enzyme product; (b) setting one or more compartments on the spot and obtaining density of the substrate in each compartment; (c) measuring the density of the enzyme product in the each compartment; and (d) estimating the enzyme activity of the enzyme based on a relationship between the density of the substrate in the each compartment obtained by step (b) and the density of the enzyme product in the each compartment obtained by step (c).

### TECHNICAL FIELD

The embodiments disclosed in the present description and the drawings relate to an apparatus for measuring enzyme activity using a substrate-immobilized carrier with non-uniform substrate immobilization density and a method thereof.

### BACKGROUND ART

The catalytic action of an enzyme on a substrate, i.e., the magnitude of enzyme activity, is evaluated by the rate v at which a reactant (enzyme product) is produced from the substrate in the presence of the enzyme. The production rate v varies depending on the substrate concentration, and reaches a maximum value when the substrate concentration is sufficiently high. Therefore, the enzyme activity of an enzyme against a substrate is generally evaluated based on two indices, namely, the maximum production rate Vmax and the affinity constant (Michaelis constant: Km), etc. in accordance with the Michaelis-Menten equation.

A common method for determining indices (Km, Vmax, etc.) of the enzyme activity of a specific enzyme against a substrate is as follows: determining the production rate of the enzyme product (enzyme product concentration/reaction time) by adding a constant concentration of the enzyme to a plurality of substrate solutions having different concentrations of the substrate, then fitting resulting values to the Michaelis-Menten equation to determine Km and the maximum production rate v of the enzyme product, which is equated with Vmax, and thus determining the enzyme activity. This method is effective when the product has luminescence, fluorescence, light absorbance, or the like, which allows direct measurement of the product. However, a product having difficulty in direct measurement may require a labeling procedure, which causes a problem of difficulty in performing B/F separation.

Methods for measuring enzyme activity include a method of immobilizing a substrate on a carrier in advance and reacting with the enzyme. This method is advantageous to a labeling procedure required for detection of an enzyme product from an enzyme reaction with respect to easy B/F separation of the labeled substance. The immobilization of a substrate is generally performed by bringing a substrate solution into contact with a solid phase. This method can be used, for example, to prepare spots of the immobilized substrate on a carrier and react them with an enzyme.

Since the enzyme reaction occurs at the solid-liquid interface in the immobilized substrate, the reaction between the immobilized substrate and the enzyme requires (i) equalizing the immobilized area of each spot to maintain a constant enzyme supply rate for each spot, and (ii) forming spots having different amounts of the immobilized substrate.

Although conventionally measures of actually equalizing the immobilized area of each spot have been taken to satisfy (i), the method of immobilizing by bringing a substrate liquid into contact with a base has problems of changing viscosity and surface tension of the liquid depending on the amount of the substrate contained in the substrate liquid, which makes it difficult to equalize the immobilized areas of the spots, particularly on a continuous carrier such as a base of slide glass. In order to equalize the immobilized areas of the spots, patterning of the base (e.g., hydrophilic/hydrophobic surfaces, patterning with or without linker substances, etc.) is usually performed before immobilization, which causes a time-consuming problem.

Although conventionally a method of bringing a known amount of a substrate solution with a known concentration into contact with a carrier has been used to satisfy (ii), the amount of the substrate immobilized on the carrier is affected by variations in multiple factors (e.g., concentration of the substrate solution, amount of spotting solution, density of the linker substance on the carrier, binding efficiency of the substrate to the carrier, etc.), and thus the immobilized amount of the substrate is not necessarily the same value as expected at the time of spotting, which may cause spots having the expected amount of the substrate to be unavailable. In addition, preparation of multiple immobilized spots with different amounts of a substrate on a solid phase carrier having a finite area causes a limited number of spots that can be prepared, and also there is a problem of time and effort of preparing substrate solutions with different concentrations to form spots.

### CITATION LIST

### Patent Literature

[PTL 1] JP-A-2000-325086

### SUMMARY OF INVENTION

### Technical Problem

One of the problems to be solved by the embodiments disclosed in this description and the drawings is to enable simple measurement of enzyme activity. However, the problems to be solved by the embodiments disclosed in this description and the drawings are not limited to the above problem. The problems corresponding to the effects of the configurations shown in the embodiments described below can also be regarded as other problems.

### Solution to Problem

The method for measuring enzyme activity according to the embodiment is a method for estimating enzyme activity using a base on which spots of an immobilized substrate are formed. This method comprises the steps: (a) allowing an enzyme to act on a substrate on a spot to obtain an enzyme product; (b) setting one or more compartments on the spot and obtaining the density of the substrate in each compartment; (c) measuring the density of the enzyme product in each compartment; and (d) estimating the enzyme activity of the enzyme based on a relationship between the density of the substrate in each compartment obtained by step (b) and the density of the enzyme product in each compartment obtained by step (c).

The apparatus for measuring enzyme activity according to the embodiment is an information processing apparatus that estimates enzyme activity using a base on which spots of immobilized substrates are formed. The apparatus comprises a means for enabling an enzyme to act on a substrate on a spot to obtain an enzyme product, a means for setting one or more compartments on the spot and obtaining the density of the substrate in each compartment, a means for measuring the density of the enzyme product in each compartment, and a means for estimating the enzyme activity of the enzyme based on a relationship between the density of the substrate in each compartment and the density of the enzyme product in each compartment.

### BRIEF DESCRIPTION OF DRAWINGS

FIG. 1 shows an example of a fluorescent image of spots on a protein array plate where a substrate is immobilized at non-uniform immobilization density.

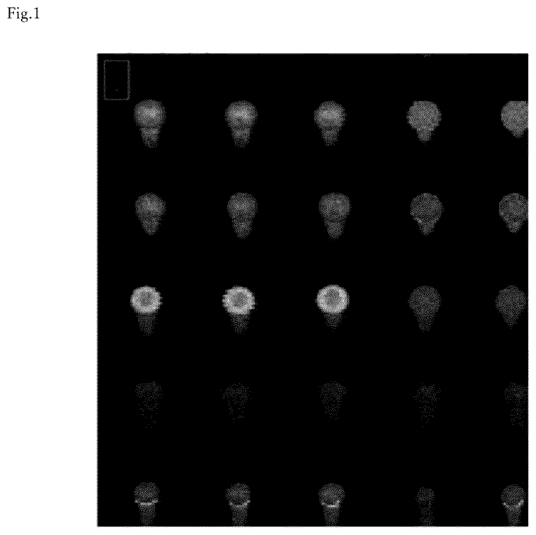

FIG. 2 is a diagram showing an example of a flow of the method for measuring enzyme activity according to the first embodiment.

FIG. 3 is a schematic diagram of the procedure for phosphorylating (for 2 hours)/labeling multiple compartments (spatially independent spots) containing different amounts of an immobilized substrate (Src), and shows fluorescent images of the compartments after the procedure.

FIG. 4 shows, with respect to the compartments (spatially independent spots) containing different amounts of an immobilized substrate (Src), fluorescent images and a graph plotting the density value (I785 nm) of the enzyme product as a function of the density value (I670 nm) of the substrate in each compartment after phosphorylating (for 2 hours)/labeling.

FIG. 5 shows, with respect to multiple compartments (virtual compartments set to be 1 pixel (10×10 μm) per compartment within one spot) containing different amounts of an immobilized substrate (Src), a schematic diagram of the procedure for a graph plotting of the density value of the enzyme product (I785 nm) as a function of the density value of the substrate (I670 nm) in one compartment after phosphorylating (for 2 hours)/labeling.

FIG. 6 shows, with respect to multiple compartments (virtual compartments set to be 1 pixel (10×10 μm) per compartment within one spot) containing different amounts of an immobilized substrate (Src), a graph plotting the density value (I785 nm) of the enzyme product as a function of the density value (I670 nm) of the substrate in each compartment after phosphorylating (for 2 hours)/labeling.

FIG. 7 shows a fluorescent image after applying smoothing (mean filter) of a spot in which each compartment is set to 1 pixel (10×10 μm).

FIG. 8 shows, with respect to multiple compartments (virtual compartments set to be 1 pixel (10×10 μm) per compartment within one spot) containing different amounts of an immobilized substrate (Src) on the fluorescent images which are processed with mean filter, a graph plotting the density value (I785 nm) of the enzyme product as a function of the density value (I670 nm) of the substrate in each compartment after phosphorylating (for 2 hours)/labeling.

FIG. 9 shows, with respect to multiple compartments (virtual compartments set to be 1 pixel (10×10 μm) per compartment within one spot) containing different amounts of an immobilized substrate (HCK), a graph plotting the density value (I785 nm) of the enzyme product as a function of the density value (I670 nm) of the substrate in each compartment after phosphorylating (for 2 hours)/labeling.

FIG. 10 shows, with respect to multiple compartments (virtual compartments set to be 1 pixel (10×10 μm) per compartment within one spot) containing different amounts of an immobilized substrate (ABL1), a graph plotting the density value (I785 nm) of the enzyme product as a function of the density value (I670 nm) of the substrate in each compartment after phosphorylating (for 2 hours)/labeling.

### DESCRIPTION OF EMBODIMENTS

Hereinafter, embodiments of the apparatus for measuring enzyme activity and the method for measuring enzyme activity will be described in detail with reference to the drawings.

### First Embodiment

The method for measuring enzyme activity according to the first embodiment is a method for estimating enzyme activity using a base on which spots of an immobilized substrate are formed. This method comprises the steps: (a) enabling an enzyme to act on the substrate on the spot to obtain an enzyme product; (b) setting one or more compartments on the spot and obtaining the density of the substrate in each compartment; (c) measuring the density of the enzyme product in each compartment; and (d) estimating the enzyme activity of the enzyme based on a relationship between the density of the substrate in each compartment obtained by step (b) and the density of the enzyme product in each compartment obtained by step (c).

The method for measuring enzyme activity according to the first embodiment is a method for estimating enzyme activity using a base on which spots of an immobilized substrate are formed.

The “base” in the method for measuring enzyme activity according to the first embodiment means a carrier for immobilizing a substrate, and includes, but is not limited to, a two-dimensional plate, a polymer gel, a fiber and a fiber sheet, beads, a rod, etc. The surface of the “base” may be smooth, and may also have a micro/nano structure such as a porous structure or fiber. The “base” is preferably a two-dimensional plate, and examples of the two-dimensional plate include plate-like base such as a slide glass and a cover glass, and well base such as an array plate. The “base” is more preferably a well base such as an array plate. The array plate has spots for containing a substrate and is used for comprehensive analysis of samples, and is also called a microchip, a microarray, a protein chip, a DNA chip, etc.

The “substrate” in the method for measuring enzyme activity according to the first embodiment is not particularly limited as long as it is a substance that satisfies both of the requirements that it can be physically or chemically immobilized on a base and that there is a means for measuring the amount of the “substrate”, wherein specific examples thereof include proteins, peptides, nucleic acids, glycans, and glycoproteins. The “substrate” may be immobilized on the base in a form directly bonded thereto, or may be bonded thereto via a linker substance, or may be immobilized in a form encompassed by a gel substance.

The “spot” in the method for measuring enzyme activity according to the first embodiment means a fixed closed area where a substrate is immobilized on a base. The shape of the “spot” is not particularly limited and may be, for example, a square, a rectangle, a circle, an ellipse, etc., and a circle is preferred. The “spot” may be continuous or discontinuous in a two-dimensional plane. In addition, in the “spot”, the substrate may be immobilized two-dimensionally or three-dimensionally depending on the base. One type of substrate is preferably immobilized on a “spot”, and two or more types of substrates may be immobilized on the same area as long as their types and their corresponding enzyme products can be distinguished and measured. According to a preferred aspect of the first embodiment, a “spot” is a circular area where one type of substrate is immobilized on a two-dimensional plate.

The method for measuring enzyme activity according to the first embodiment includes a step of (a) enabling an enzyme to act on a substrate on a spot to obtain an enzyme product.

The “enzyme product” in the method for measuring enzyme activity according to the first embodiment refers to a substance obtained as a result of an enzyme reacting with a substrate. Such enzyme products include any of those produced by an addition reaction of a substrate, those produced by a substitution reaction of a substrate, and those produced by a cleavage reaction in which a portion of the substrate has been cleaved (the cleavage site is removed), or combinations thereof. The “enzyme” in the method for measuring enzyme activity according to the first embodiment is not particularly limited, and includes all substances that catalyze addition reactions, substitution reactions, cleavage reactions, or combinations thereof. Examples of the “enzyme” in the method for measuring enzyme activity according to the first embodiment include proteases, kinases, oxidases, nucleases, etc., and preferably kinases.

The method for measuring enzyme activity according to the first embodiment includes a step of (b) setting one or more compartments on the spot and obtaining the density of the substrate in each compartment.

In the “spot” of the method for measuring enzyme activity according to the first embodiment, the substrate may be uniformly or non-uniformly immobilized. Here, the term “the substrate is uniformly immobilized” means that substantially uniform immobilization is sufficient, and does not mean that inclusion of non-uniform density regions is not permitted. Furthermore, in a “spot” in the method for measuring enzyme activity according to the first embodiment, the substrate may be uniformly immobilized in some region and non-uniformly immobilized in another region.

In the method for measuring enzyme activity according to the first embodiment, a closed region in which the density of the immobilized substrate is uniform is defined as a “compartment”. Here, the term “uniform density of the immobilized substrate” means that substantially uniform density is sufficient, and does not mean that inclusion of a region with non-uniform density is not permitted. The density of the immobilized substrate in a “compartment” may be the same as or different from that in other “compartments.” Furthermore, a “compartment” may be continuous or discontinuous with respect to other “compartments” in a two-dimensional plane. The shape of the “compartment” is not particularly limited, and may be, for example, a square, a rectangle, a circle, an ellipse, etc.

In the method for measuring enzyme activity according to the first embodiment, the “uniform density of the immobilized substrate” may be the average density in one compartment.

According to a preferred first embodiment, the compartments are selected from spatially independently arranged spots. In this case, the spatially independently arranged spot is considered to be one unit of the compartment, and corresponding to this is, for example, a spatially independently arranged spot having uniform density of the immobilized substrate.

According to another preferred first embodiment, the compartment is selected from a closed area, which is smaller than a spot and a plurality of which can be set within a spot, having uniform density of the immobilized substrate. Here, the term “uniform density of the immobilized substrate” means that substantially uniform density is sufficient, and does not mean that inclusion of a region with non-uniform density is not permitted. Such a single closed area is called a “virtual compartment,” and the “virtual compartment” is regarded as one unit of the compartment. The size and shape of the “virtual compartment” may be determined arbitrarily. A “virtual compartment” is the same as a “compartment” in that the density of the immobilized substrate may be the same as or different from that of other “virtual compartments.” Furthermore, a “virtual compartment” may be continuous or discontinuous with respect to other “virtual compartments” in a two-dimensional plane. The shape of the “virtual compartment” is not particularly limited, and may be, for example, a square, a rectangle, a circle, an ellipse, etc. An example of a “virtual compartment” includes a compartment set in the form of a square of about 1 pixel (10 μm×10 μm) in a spot which has a size of several hundred μm² and includes areas having different values of the density of the immobilized substrate.

In the method for measuring enzyme activity according to the first embodiment, the uniform density of the immobilized substrate may be the average density in one virtual compartment.

In the method for measuring enzyme activity according to the first embodiment, the method for producing spots having non-uniform values of the density of immobilized substrate is not particularly limited, and can be performed in accordance with known methods, for example, by tilting the base, using the coffee ring effect, changing the temperature (convection control), changing the immobilization density of the linker substance of the substrate, pinning the liquid by a contact method, spotting in multiple stages, spotting substrate liquids of different concentrations at different points, etc.

In the method for measuring enzyme activity according to the first embodiment, the density of a substrate in a compartment may be obtained by measuring the amount of the substrate in the compartment and dividing it by the area of the compartment, or if the value is known, the value may be taken. The amount of the substrate in a compartment can be measured by a known method, for example, quantification by SPR (surface plasmon resonance) method, quantification based on electrochemical properties (e.g., potential, current value, impedance, capacitance, etc.), quantification based on the presence distribution of elements obtained by X-ray spectroscopy etc., quantification using AFM, quantification based on signal information derived from a labeling substance introduced into the substrate, etc., and quantification based on signal information derived from a labeling substance introduced into the substrate is preferred. The labeling substance is not particularly limited and is preferably optically detectable, and examples thereof include fluorescent substances, light-absorbing substances, light-emitting substances, scattering substances, polarizing substances, and oxidation-reduction substances.

The method for measuring enzyme activity according to the first embodiment includes a step of (c) measuring the density of the enzyme product in each compartment.

In the method for measuring enzyme activity according to the first embodiment, the density of an enzyme product in a compartment can be obtained by measuring the amount of the enzyme product in the compartment and dividing it by the area of the compartment. The amount of the enzyme product in a compartment can be measured by known methods, for example, quantification by SPR (surface plasmon resonance) method, quantification based on electrochemical properties (e.g., potential, current value, impedance, capacitance, etc.), quantification based on the presence distribution of elements obtained by X-ray spectroscopy, quantification using AFM, quantification based on signal information derived from a labeling substance introduced into the enzyme product, etc., and quantification based on signal information derived from a labeling substance introduced into the enzyme product is preferred. The labeling substance is not particularly limited and is preferably optically detectable, and examples thereof include fluorescent substances, light-absorbing substances, light-emitting substances, scattering substances, polarizing substances, and oxidation-reduction substances.

In the method for measuring enzyme activity according to the first embodiment, the density of the enzyme product in each compartment may be the average density in one compartment.

The method for measuring enzyme activity of the first embodiment includes a step of (d) estimating the enzyme activity based on a relationship between the density of the substrate in each compartment obtained by step (b) and the density of the enzyme product in each compartment obtained by step (c).

In the method for measuring enzyme activity according to the first embodiment, estimation of the enzyme activity is performed by first calculating the maximum production rate (Vmax) of the enzyme product based on a relationship between the density of the substrate and the density of the enzyme product in each compartment, and then estimating the enzyme activity based on the obtained value of the maximum production rate of the enzyme product. The estimation of the enzyme activity may be performed further taking into consideration the value of the affinity constant (Michaelis constant: Km), and other parameters necessary for the estimation may also be taken into consideration, if any.

As described above, in the method for measuring enzyme activity according to the first embodiment, the enzyme activity is estimated by first calculating the maximum production rate of the enzyme product based on a relationship between the density of the substrate and the density of the enzyme product in each compartment, and then calculating the enzyme activity based on the obtained value of the maximum production rate of the enzyme product. Therefore, the method for measuring enzyme activity according to the first embodiment may be a method for measuring the maximum production rate of an enzyme product.

Such a method for measuring the maximum production rate is a method for measuring the maximum production rate of an enzyme product using a base on which spots of immobilized substrates are formed. This method comprises the steps: (a) enabling an enzyme to act on a substrate on a spot to obtain an enzyme product; (b) setting one or more compartments on the spot and obtaining the density of the substrate in each compartment; (c) measuring the density of the enzyme product in each compartment; and (d) calculating the maximum production rate of the enzyme product based on a relationship between the density of the substrate in each compartment obtained by step (b) and the density of the enzyme product in each compartment obtained by step (c).

The maximum production rate value can be calculated, for example, based on the results of plotting on a graph the density value of the enzyme product in each compartment or the value obtained by dividing the density value by the reaction time as a function of the density value of the substrate in each compartment. Incidentally, the relationship between these values may be fitted using the Michaelis-Menten equation. Therefore, the estimation of enzyme activity and the calculation of the maximum production rate preferably include a step of fitting using the Michaelis-Menten equation. When fitting using the Michaelis-Menten equation is performed, the affinity constant of the substrate for the enzyme may further be calculated. In addition, instead of fitting using the Michaelis-Menten equation, an equation obtained by taking the reciprocal of the Michaelis-Menten equation (Lineweaver-Burk equation) can also be used.

Fitting by the Michaelis-Menten equation, without considering the decrease in the reaction rate due to a decrease in the amount of unreacted substrate during the enzyme reaction, can be performed, for example, by conducting an enzyme reaction for a certain reaction time, determining the density of the substrate and the density of the enzyme product in each compartment, then obtaining a graph with the density of the enzyme product converted into the production rate by dividing by the reaction time, and fitting by the Michaelis-Menten equation on the graph to calculate the value of the maximum production rate. In addition, the density of the substrate corresponding to a production rate of a half of the maximum production rate can be calculated as an affinity constant.

When considering a decrease in the reaction rate due to a decrease in the amount of unreacted substrate during the enzyme reaction, the above calculation can be substituted, for example, with the following: (i) assuming a certain affinity constant and a maximum production rate, and determining the production rate of the enzyme product at the start of the reaction (t=0) for the density of the substrate in each compartment from the Michaelis-Menten equation; (ii) determining the amount of the enzyme product produced and the amount of decrease in the amount of the unreacted substrate after a unit time has elapsed, and also determining the production rate of the enzyme product corresponding to the unreacted substrate density at t=unit time; (iii) repeating (i) and (ii) to obtain an evaluation result of the relationship between the density of the substrate and the density of the enzyme product when a specified reaction time has elapsed; and (iv) converging the maximum production rate and the affinity constant so as to minimize the error between the evaluation result obtained in (iii) and the actual measurement result.

In the method for measuring enzyme activity according to the first embodiment, the “specimen” containing the enzyme as a measuring object can be determined appropriately based on the purpose by a person performing the method, and anything containing an enzyme can be a “specimen”. The “specimen” includes biologically derived substances, extracts from biological bodies, blood, blood-derived substances, food, food-derived substances, natural products, substances derived from natural products, and substances derived from culture medium. The “specimen” may be pretreated as appropriate depending on the purpose and procedure, or a reagent may be added thereto beforehand. The “specimen” may be in the form of gas, solid, or liquid, and is appropriately used in liquid form by diluting, suspending, or extracting in water, physiological saline, a buffer solution, or other solution. The specimen may contain preservatives and other additives. In addition, reagents are added to the “specimen” depending on the purpose.

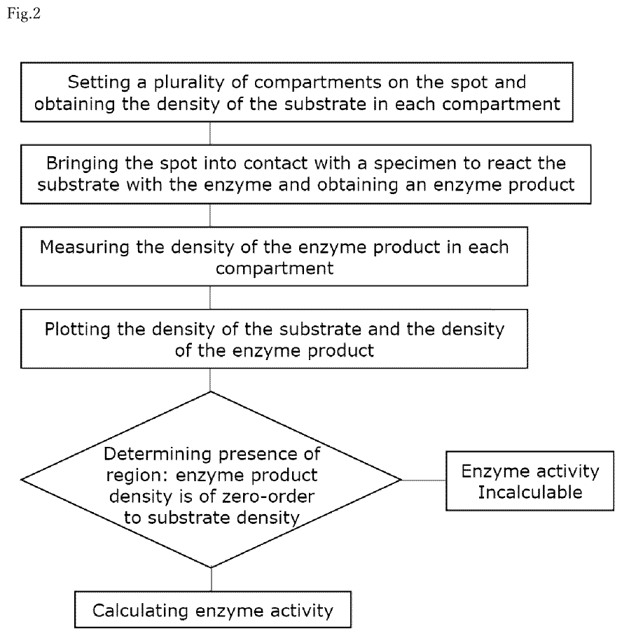

FIG. 2 is a diagram showing an example of a flow of the method for measuring enzyme activity according to the first embodiment. The method for measuring enzyme activity shown in FIG. 2 includes the following steps: setting a plurality of compartments on a spot and obtaining the density of the substrate in each compartment; bringing the spot into contact with a specimen to react the substrate with the enzyme and obtaining the enzyme product; measuring the density of the enzyme product in each compartment; plotting on a graph the density value of the enzyme product or a value obtained by dividing it by the reaction time in each compartment as a function of the density value of the substrate in each compartment to determine the presence of a region in which the density value of the enzyme product in each compartment or the value obtained by dividing the density value by the reaction time is of zero-order with respect to the density value of the substrate in each compartment; and when a zero-order region is present, calculating the enzyme activity value of the enzyme based on the above function, and when a zero-order region is not present, determining that the calculation of enzyme activity of the enzyme is impossible. This method enables calculation of enzyme activity using a base on which spots of immobilized substrates are formed. Furthermore, in this method, the density of the substrate in each compartment may be obtained at any timing before the step of plotting on a graph the density value of the enzyme product or a value obtained by dividing it by the reaction time in each compartment as a function of the density value of the substrate in each compartment to determine the presence of a region in which the density value of the enzyme product in each compartment or the value obtained by dividing the density value by the reaction time is of zero-order with respect to the density value of the substrate in each compartment.

The method for measuring enzyme activity shown in FIG. 2 includes a step of setting a plurality of compartments on a spot and obtaining the density of the substrate in each compartment. The density of the substrate may be obtained by measurement, and when the value is known, it may be used.

The method for measuring enzyme activity shown in FIG. 2 includes a step of bringing the spot into contact with a specimen to react the substrate with the enzyme and obtaining the enzyme product.

The method for measuring enzyme activity shown in FIG. 2 includes a step of measuring the density of the enzyme product in each compartment.

The method for measuring enzyme activity shown in FIG. 2 includes a step of plotting on a graph the density value of the enzyme product or a value obtained by dividing it by the reaction time in each compartment as a function of the density value of the substrate in each compartment to determine the presence of a region in which the density value of the enzyme product in each compartment or the value obtained by dividing the density value by the reaction time is of zero-order with respect to the density value of the substrate in each compartment.

The zero-order region refers to a region in which increase in the substrate value on the graph does not change the value of the enzyme product (or the value obtained by dividing the value by the reaction time), which remains constant. Usually, in the measurement, increase in the amount of enzyme product corresponding to the increase in the amount of the substrate (or the increase in the value obtained by dividing the increase by the reaction time) (slope) will gradually decrease and finally become zero-order. Therefore, the zero-order region includes “a region in which the increase in the amount of the enzyme product (or the value obtained by dividing the value by the reaction time) corresponding to the increase in the amount of the substrate on the graph has a slope smaller than that of the region (seen in regions with low substrate amounts) in which linearly occurs the increase in the amount of the enzyme product (or the value obtained by dividing the value by the reaction time) corresponding to the increase in the amount of the substrate on the graph.”

The absence of a zero-order region refers to a case where there is a constant slope between the amount of the substrate and the amount of the enzyme product (or the value obtained by dividing the value by the reaction time) within the range of the measured amount of the substrate, and no change is observed in the slope of the increase in the amount of the enzyme product (or the value obtained by dividing the value by the reaction time) with respect to the increase in the amount of the substrate.

The method for measuring enzyme activity shown in FIG. 2 includes a step of, when a zero-order region is present, calculating the enzyme activity value of the enzyme based on the above function, and when zero-order region is not present, determining that the calculation of enzyme activity of the enzyme is impossible.

In the method for measuring enzyme activity shown in FIG. 2, the maximum production rate (Vmax) of the enzyme product is first calculated based on the above function, and then the enzyme activity value is calculated based on the obtained value of the maximum production rate of the enzyme product. The value of enzyme activity may be calculated further taking into consideration the value of the affinity constant (Michaelis constant: Km), and may also be calculated taking into consideration other parameters necessary for calculation, if any.

As described above, in the method for measuring enzyme activity shown in FIG. 2, the value of the maximum production rate of the enzyme product is first calculated based on the above function, and then the value of the enzyme activity is calculated based on the obtained value of the maximum production rate of the enzyme product. Therefore, the method for measuring enzyme activity shown in FIG. 2 may also be used as a method for measuring the maximum production rate of the enzyme product.

Such a method for measuring the maximum production rate includes the following steps: setting a plurality of compartments on a spot and obtaining the density of the substrate in each compartment; bringing the spot into contact with a specimen to react the substrate with the enzyme and obtaining the enzyme product; measuring the density of the enzyme product in each compartment; plotting on a graph the density value of the enzyme product or a value obtained by dividing it by the reaction time in each compartment as a function of the density value of the substrate in each compartment to determine the presence of a region in which the density value of the enzyme product in each compartment or the value obtained by dividing the density value by the reaction time is of zero-order with respect to the density value of the substrate in each compartment; and when a zero-order region is present, calculating the maximum production rate of the enzyme product based on the above function, and when zero-order region is not present, determining that the calculation of the maximum production rate of the enzyme product is impossible. This method allows calculation of the maximum production rate using a base on which spots of immobilized substrates are formed. In this method, the density of the substrate in each compartment may be obtained at any timing before the step of plotting on a graph the density value of the enzyme product or the value obtained by dividing it by the reaction time in each compartment as a function of the density value of the substrate in each compartment to determine the presence of a region in which the density value of the enzyme product in each compartment or the value obtained by dividing the density value by the reaction time is of zero-order with respect to the density value of the substrate in each compartment.

### Second Embodiment

The apparatus for measuring enzyme activity according to the second embodiment is an information processing apparatus that estimates enzyme activity using a base on which spots of immobilized substrates are formed. The apparatus comprises a means for obtaining an enzyme product by enabling an enzyme to act on the substrate on the spot, a means for obtaining the density of the substrate in each compartment by setting one or more compartments on the spot, a means for measuring the density of the enzyme product in each compartment, and a means for estimating the enzyme activity of the enzyme based on a relationship between the density of the substrate in each compartment and the density of the enzyme product in each compartment.

The meanings and references of the terms “substrate,” “spot,” “base,” “compartment,” etc. used to explain the second embodiment are the same as those used to explain the first embodiment.

The apparatus for measuring enzyme activity according to the second embodiment may further include a means for changing the measurement point. The means for changing the measurement point is not particularly limited, and includes a scanning method, a method of integrating the measurement means, etc. The scanning method may, for example, include moving the spot side and/or the measuring means side. Examples of the method for integrating the measuring means include optical sensors such as PMT, CMOS, CCD, and SPAD, and electrochemical sensors such as integrated FET.

The amounts of a substrate and an enzyme product can be measured, for example, by labeling the common site of the substrate (a site keeping unchanged structure before and after the enzyme reaction, serving as an indicator of the amount of the substrate) and the enzyme product-specific site (a structural site present only after the enzyme reaction, serving as an indicator of the enzyme product) with different fluorescent substances, obtaining the fluorescence intensity at each point with a confocal unit, and scanning each point within the spot with the above fluorescence measurement.

Enzyme activity can be easily measured in accordance with at least one of the embodiments described above.

Although several embodiments have been described, these embodiments are presented as examples and are not intended to limit the scope of the invention. These embodiments can be implemented in various other forms, and various omissions, substitutions, modifications, and combinations of embodiments can be made without departing from the spirit of the invention. These embodiments and their variations are included in the scope equivalent to that of the invention described in the scope of claims as well as in the scope and spirit of the invention.

### EXAMPLES

The present invention will be specifically described based on the following examples, but the present invention is not limited to these examples. Unless otherwise specified, the contents are expressed in mass %.

### Estimation of Maximum Phosphorylation Rate of Kinases on Immobilized Src

This example describes the following: various kinases contained in a cell extract of the human pulmonary adenocarcinoma cell line PC9 are set as the kinases to be measured, and estimated is the maximum phosphorylation rate when these kinases phosphorylate immobilized Src.

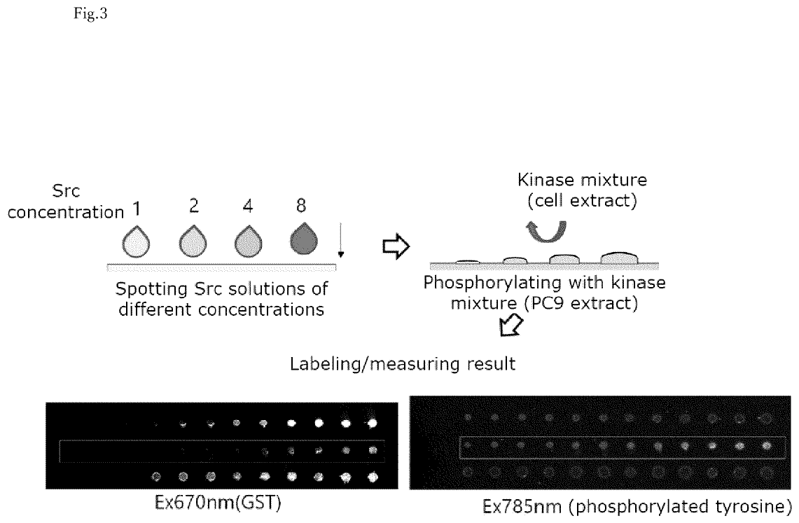

A GST (glutathione-S-transferase) tag-fused Src solution was dropped onto a glutathione (GSH)-coated slide glass prepared by the method described in non-patent literature (Tadashi Manabe et al., “IGF2 Autocrine-Mediated IGF1R Activation Is a Clinically Relevant Mechanism of Osimertinib Resistance in Lung Cancer”, Mol Cancer Res. 2020 April; 18(4):549-559.), as three spots for each Src concentration ratio of 1, 2, 4, and 8, to obtain an array plate having spots (diameter approximately 100 μm) of immobilized Src. Furthermore, a cell extract from the human pulmonary adenocarcinoma cell line PC9 was mixed with a kinase reaction solution (25 mM Tris-HCl, 5 mM β-glycerophosphate, 0.1 mM Na3VO4, 10 mM MgCl2, 1 mM ATP, and 2 mM DTT), brought into contact with the Src-immobilized spots, and incubated at 30° C. for 2 hours to induce phosphorylation on the spots. The reaction was then stopped by bringing a reaction stop solution (50 mM EDTA, 10 mM HEPES-NaOH [pH 7.4], 150 mM NaCl, and 0.05% [v/v] Tween 20) into contact with the immobilized spots and incubating at 30° C. for 5 minutes. After washing the array plate with TBST, a primary antibody reaction solution (a cocktail of mouse anti-phosphotyrosine antibody and rabbit anti-GST antibody) was added and incubated at 30° C. for 1 hour. After washing the array plate with TBST, secondary antibody solution (a cocktail of Goat anti-Mouse IgG (H+L) Highly Cross-Adsorbed Secondary Antibody, Alexa Fluor 790 (Invitrogen) and Goat anti-Rabbit IgG (H+L) Highly Cross-Adsorbed Secondary Antibody, Alexa Fluor 680 (Invitrogen)) was added and incubated at 30° C. for 1 hour, and thus the phosphorylated tyrosine generated in the immobilized spots of Src was labeled with Alexa Fluor 790, and the GST tag in Src was labeled with Alexa Fluor 680. The labeled array plate was measured using a microarray scanner having a confocal optical system to obtain a fluorescent image. Alexa Fluor 790 was measured using an excitation laser with a wavelength of 785 nm (the fluorescence signal for this is shown as 1785 nm), and Alexa Fluor 680 was measured using an excitation laser with a wavelength of 670 nm (the fluorescence signal for this is shown as 1670 nm) (FIG. 3). From the obtained fluorescent images, the maximum phosphorylation rate of Src was obtained by the following two methods.

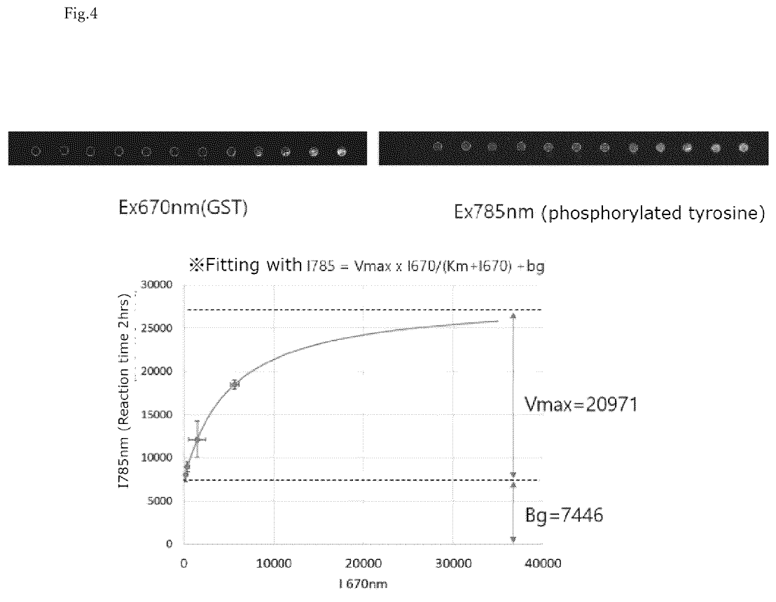

In the first method, each spot, as a compartment for determining the density of Src and the density of the enzyme product, was spatially arranged independently on the plate, and the density value (I785 nm) of the enzyme product (phosphorylated tyrosine) in each spot was plotted as a function of the density value (I670 nm) of the substrate in each spot (FIG. 4). In the plot, plotted were the average values and standard deviations of 1785 nm and 1670 nm obtained for each of three spots prepared by dropping Src solutions of the same concentration. Each spot was identified as a region with the intensity of 1670 nm exceeding a certain threshold. The plotting was performed by fitting using the Michaelis-Menten equation considering the presence of background (Bg) (1)

$$\begin{matrix}[ Math . 1 ] & \\ {I}_{785 nm} = \frac{{V}_{max} {I}_{670 nm}}{{K}_{m} + {I}_{670 nm}} + Bg & ( 1 )\end{matrix}$$

to obtain the maximum phosphorylation rate after a reaction time of 2 hours of Vmax(/2 h)=20971, the Michaelis constant (affinity constant) Km=5031, and Bg=7446.

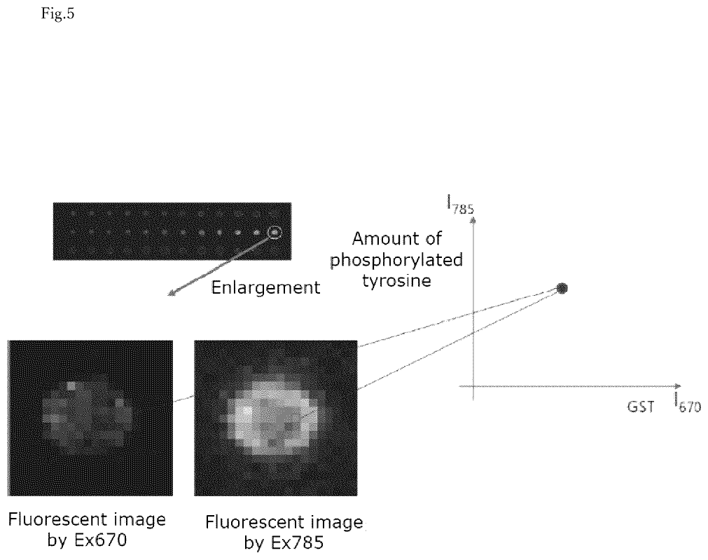

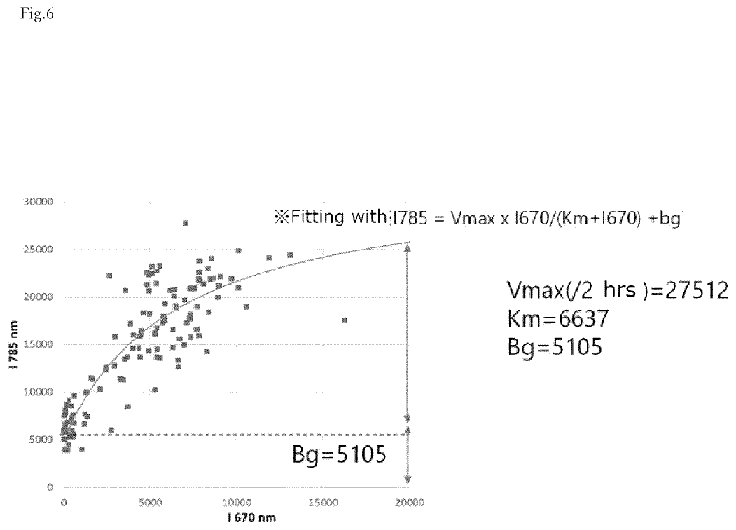

In the second method, a virtual compartment of 1 pixel (10×10 μm), which is smaller than one spot, was set as the compartment for determining the density of Src and the density of the enzyme product. The density value (I785 nm) of the enzyme product (phosphorylated tyrosine) in one virtual compartment (1 pixel) set within one spot (the rightmost spot in FIG. 5 was plotted as a function of the density value (I670 nm) of the substrate in the same compartment (FIG. 5), and this operation was repeated throughout the entire spot to obtain a group of plots of the substrate density and the enzyme product density. By fitting the obtained plot group using the above-mentioned formula (1), obtained were the maximum phosphorylation rate after a reaction time of 2 hours, Vmax(/2 h)=27512, Michaelis constant (affinity constant) Km=6637, and Bg=5105 (FIG. 6). The reason for the slight difference in values between the first and second methods was presumably that the distribution of substrate density values (I670 nm) used for fitting was slightly narrower in the first method.

In the second method, the local substrate density variation within a single spot is used as the distribution of values of the substrate density among virtual compartments, thereby obtaining density information of the enzyme product over a wider range of substrate density distributions. In addition, the Vmax and Km values obtained here are in arbitrary units based on fluorescence brightness, and these units can also be converted into units of the substrate density and the enzyme product density by preparing a calibration curve from the fluorescence brightness obtained for compartments having known substrate density and the enzyme product density and performing the conversion.

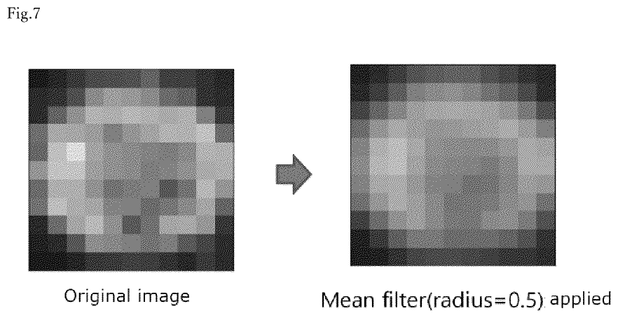

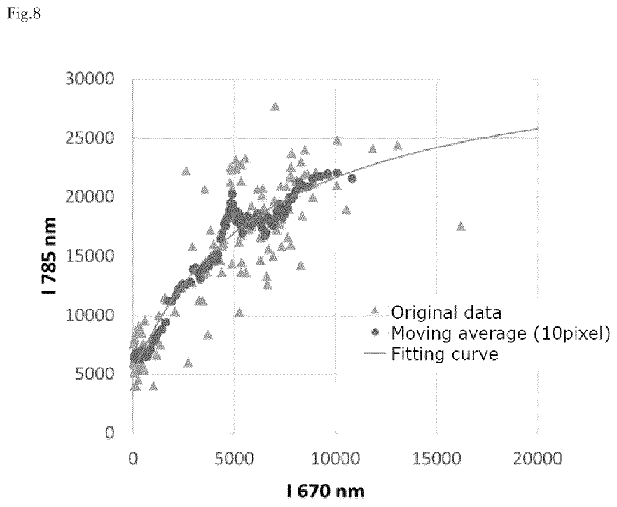

In either the first or second method, filtering can be performed on each obtained fluorescent image. The results using the Mean filter are shown in FIG. 7. Locally occurring noise components on the image can be cancelled while maintaining distribution information of the amounts of the substrate and enzyme products on the fluorescent image (FIG. 7). In addition, after determining the substrate density and the enzyme product density for each compartment, the enzyme product density can be sorted in the order of close substrate density and then subjected to a moving average. In the distribution of the substrate density and the enzyme product density for each compartment (1 pixel (10×10 μm)) obtained using the second method, the compartments were sorted in descending order of the substrate density, and then subjected to the moving average for 10 compartments, which results are shown in FIG. 8. This procedure facilitates grasping the tendency of the enzyme product to change with the substrate density, and thus the fitting procedure (FIG. 8).

### Estimation of Maximum Phosphorylation Rate of Kinases on Immobilized HCK

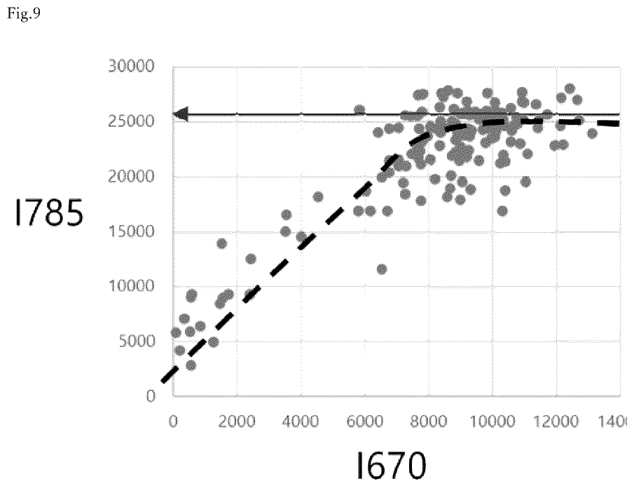

The procedure was performed in the same manner as for Src. The results are shown in FIG. 9. Incidentally, calculation of the maximum phosphorylation rate Vmax is not necessarily performed by fitting based on the Michaelis-Menten equation, and Vmax may be calculated based on the density of the enzyme product in a region where the density of the enzyme product is constant with respect to the density of the substrate.

### Estimation of Maximum Phosphorylation Rate of Kinases on Immobilized ABL1

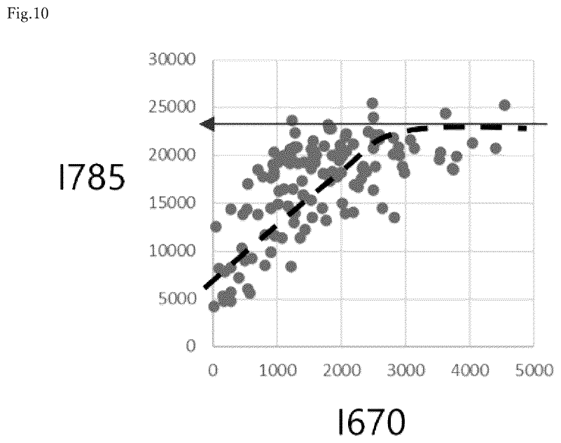

The procedure was performed in the same manner as for Src. The results are shown in FIG. 10. Incidentally, calculation of the maximum phosphorylation rate Vmax is not necessarily performed by fitting based on the Michaelis-Menten equation, and Vmax may be calculated based on the density of the enzyme product in a region where the density of the enzyme product is constant with respect to the density of the substrate.

Estimation of Maximum Reaction Rate of Each Enzyme when Multiple Enzymes Act on Multiple Substrates

Enzyme reaction rates were estimated based on the maximum reaction rates obtained with the immobilized substrate.

When one enzyme x₁ converts one substrate Y₁ to an enzyme product Y₁′, the maximum reaction rate obtained for the substrate Y₁:

$$( {V}_{max}^{Y 1 \to Y {1}^{′}} ) \qquad [ Math . 2 ]$$

was considered to be equal to the maximum reaction rate of the enzyme:

$$( {v}_{x 1 max}^{Y 1 - Y {1}^{′}} ) . \qquad [ Math . 3 ]$$

$$[ Math . 4 ]$$

$${V}_{max}^{Y 1 \to Y {1}^{′}} = {v}_{x 1 max}^{Y 1 \to Y {1}^{′}} \qquad ( 2 )$$

When one type of enzyme x₁ converts multiple types of substrates Yⱼ (j=1, 2, . . . , n) into enzyme products Yⱼ′ (j=1, 2, . . . , n), the maximum reaction rates obtained for the substrate Yⱼ:

$$( {V}_{max}^{Y 1 \to Y {1}^{′}} \cdot {V}_{max}^{Y 2 \to Y {2}^{′}} , … , {V}_{max}^{Yn \to {Yn}^{′}} ) \qquad [ Math . 5 ]$$

were considered to be equal to the maximum reaction rate of enzyme x₁ with substrate Yⱼ (j=1, 2, . . . , n):

$$( {v}_{x 1 max}^{Y 1 \to Y {1}^{′}} \cdot {v}_{x 1 max}^{Y 2 \to Y {2}^{′}} , … , {v}_{x 1 max}^{Yn \to {Yn}^{′}} ) . \qquad [ Math . 6 ]$$

$$[ Math . 7 ]$$

$$\begin{matrix}{V}_{max}^{Y 1 \to Y {1}^{′}} = {v}_{x 1 max}^{Y 1 \to Y {1}^{′}} \\ {V}_{max}^{Y 2 \to Y {2}^{′}} = {v}_{x 1 max}^{Y 2 \to Y {2}^{′}} \\ ⋮ \\ {V}_{max}^{Y n \to Y {n}^{′}} = {v}_{x 1 max}^{Y n \to Y {n}^{′}}\end{matrix} \qquad ( 3 )$$

When multiple enzymes xᵢ (i=1, 2, . . . , m) act on one or multiple substrates Yⱼ (j=1, 2, . . . , n), between the maximum reaction rates obtained for a substrate Yⱼ:

$${V}_{max}^{Yj \to {Yj}^{′}} \qquad [ Math . 8 ]$$

and the maximum reaction rate of each enzyme xᵢ for each substrate Yⱼ:

$${v}_{xi max}^{Yj \to {Yj}^{′}} , \qquad [ Math . 9 ]$$

the following simultaneous equations (4) were considered to hold true.

$$[ Math . 10 ]$$

$$\{ \begin{matrix}{V}_{max}^{Y 1 \to Y {1}^{′}} = {v}_{x 1 max}^{Y 1 \to Y {1}^{′}} + {v}_{x 2 max}^{Y 1 \to Y {1}^{′}} + … + {v}_{xm max}^{Y 1 \to Y {1}^{′}} \\ {V}_{max}^{Y 2 \to Y {2}^{′}} = {v}_{x 1 max}^{Y 2 \to Y {2}^{′}} + {v}_{x 2 max}^{Y 2 \to Y {2}^{′}} + … + {v}_{xm max}^{Y 2 \to Y {2}^{′}} \\ ⋮ \\ {V}_{max}^{Yn \to {Yn}^{′}} = {v}_{x 1 max}^{Y n \to Y {n}^{′}} + {v}_{x 2 max}^{Y n \to Y {n}^{′}} + … + {v}_{xm max}^{Y n \to Y {n}^{′}}\end{matrix} \qquad ( 4 )$$

The simultaneous equations (4) have m×n unknowns and n equations, and cannot be solved except for the above-mentioned case of m=1.

$${v}_{xi max}^{Yj - Yf} \qquad [ Math . 11 ]$$

To estimate the above, the unknowns need to be reduced by applying the ratio of the maximum reaction rates of each enzyme for each substrate.

As described above, the maximum reaction rates of one type of enzyme x₁ for multiple types of substrates Yⱼ (j=1, 2, . . . , n):

$$( {v}_{x 1 max}^{Y 1 \to Y {1}^{′}} , {v}_{x 1 max}^{Y 2 \to Y {2}^{′}} , … , {v}_{x 1 max}^{Yn \to {Yn}^{′}} ) \qquad [ Math . 12 ]$$

are equal to the maximum reaction rates obtained for substrate Yⱼ:

$$( {V}_{max}^{Y 1 \to Y {1}^{′}} , {V}_{max}^{Y 2 \to Y {2}^{′}} , … , {V}_{max}^{Yn \to {Yn}^{′}} ) , \qquad [ Math . 13 ]$$

and thus, by measuring the maximum reaction rates for each substrate when only one type of enzyme x₁ is reacted with multiple types of substrates Yⱼ (j=1, 2, . . . , n):

$$( {V}_{max}^{Y 1 \to Y {1}^{′}} , {V}_{max}^{Y 2 \to Y {2}^{′}} , … , {V}_{max}^{Yn \to {Yn}^{′}} ) , \qquad [ Math . 14 ]$$

the ratios of the maximum reaction rate of enzyme x₁ for each substrate can be obtained. By performing the same operation for the other enzymes x₂, x₃, . . . , xₙ, the ratios of the maximum reaction rate of each enzyme for each substrate can be obtained based on the actual measured values.

The ratios of the maximum reaction rates when an enzyme xᵢ (i=1, 2, . . . , m) acts on one or more substrates Yⱼ (j=1, 2, . . . , n) are obtained as:

$$[ Math . 15 ]$$

$$\begin{matrix}{v}_{x 1 max}^{Y 1 \to Y {1}^{′}} : {v}_{x 1 max}^{Y 2 \to Y {2}^{′}} : … : {v}_{x 1 max}^{Yn \to {Yn}^{′}} : … = α : β : … : γ \\ {v}_{x 2 max}^{Y 1 \to Y {1}^{′}} : {v}_{x 2 max}^{Y 2 \to Y {2}^{′}} : … : {v}_{x 2 max}^{Yn \to {Yn}^{′}} : … = δ : ε : … : ζ \\ ⋮ \\ {v}_{xm max}^{Y 1 \to Y {1}^{′}} : {v}_{x m max}^{Y 2 \to Y {2}^{′}} : … : {v}_{xm max}^{Yn \to {Yn}^{′}} : … = η : θ : … : λ\end{matrix} , \qquad ( 5 )$$

and then equation (4) can be converted as follows:

$$[ Math . 16 ]$$

$$( \begin{matrix}{V}_{max}^{Y 1 \to Y {1}^{′}} \\ {V}_{max}^{Y 2 \to Y {2}^{′}} \\ ⋮ \\ {V}_{max}^{Y n \to Y {n}^{′}}\end{matrix} ) = ( \begin{matrix}1 & 1 & … & 1 \\ \frac{β}{α} & \frac{ε}{δ} & … & \frac{θ}{η} \\ ⋮ & ⋮ & ⋱ & ⋮ \\ \frac{γ}{α} & \frac{ζ}{δ} & … & \frac{λ}{η}\end{matrix} ) ( \begin{matrix}{v}_{x 1 max}^{Y 1 \to Y {1}^{′}} \\ {v}_{x 2 max}^{Y 1 \to Y {1}^{′}} \\ ⋮ \\ {v}_{xm max}^{Y 1 \to Y {1}^{′}}\end{matrix} ) . \qquad ( 6 )$$

When n≥m (substrate type≥enzyme type), equation (6) is a simultaneous linear equation under excess conditions, and the least-square solution that minimizes the square of the distance between the left and right sides can be sought:

$${v}_{x 1 max}^{Y 1 \to Y {1}^{′}} , {v}_{x 2 max}^{Y 1 \to Y {1}^{′}} , … , {v}_{x m max}^{Y 1 \to {Y 1}^{′}} . \qquad [ Math . 17 ]$$

Finally, by referring to the ratios of the maximum reaction rate of each enzyme for each substrate in equation (5), the maximum reaction rates of each enzyme xᵢ for each substrate Yⱼ can be calculated as:

$${v}_{x 1 max}^{Y j \to Y {j}^{′}} . \qquad [ Math . 18 ]$$

### Dividing Areas in a Spot Between Substrate Amount Rate-Limiting/Enzyme Reaction Rate-Limiting

After setting within a single spot imaginary compartments smaller than the spot, determining and plotting the substrate density and the enzyme product density in each compartment allows division of areas between a region where the density of the enzyme product increases with increasing substrate density and a region where the density of the enzyme product is constant (zero-order) in spite of increasing substrate density. In this case, the former is regarded as a region of substrate amount rate-limiting, and the latter is regarded as a region of enzyme reaction rate-limiting. In such a spot, a region where an enzyme reaction is performed at the maximum reaction rate is a region of enzyme reaction rate-limiting, and in the enzyme reaction rate-limiting region, the density of the enzyme product is proportional to the maximum reaction rate of the enzyme. In contrast, estimation of the affinity constant Km requires the presence of a region of substrate amount rate-limiting. When a certain substrate has an extremely small (e.g., less than 1%) region of either substrate amount rate-limiting or enzyme reaction rate-limiting, estimation of the maximum reaction rate and the affinity constant based on the substrate density and the enzyme product density in such a small region results in large estimation errors due to susceptibility to the influence of noise components.

On the basis of the above, applications of comparison and analysis of enzyme activity based on the density of the enzyme product allow exhibition to the user dividing areas in a spot into substrate amount rate-limiting/enzyme reaction rate-limiting. The methods of exhibition may include displaying each region superimposed on the image of the spot, or showing the proportion of each region for each spot, for each type of the substrate, or for each plate. Alternatively, the following can be performed: as a result of the dividing, the apparatus determines whether the substrate amount rate-limiting/enzyme reaction rate-limiting region has a sufficient area, and in the case of an insufficient area, displayed to the user is an error message that the estimated enzyme activity indicator may be unreliable.

1. A method for estimating enzyme activity using a base on which spots of immobilized substrates are formed, comprising the steps:
(a) enabling an enzyme to act on the substrate on the spot to obtain an enzyme product;
(b) setting one or more compartments on the spot and obtaining density of the substrate in each compartment;
(c) measuring the density of the enzyme product in the each compartment; and
(d) estimating the enzyme activity of the enzyme based on a relationship between the density of the substrate in the each compartment obtained by step (b) and the density of the enzyme product in the each compartment obtained by step (c).

2. The method according to claim 1, wherein the compartment is a single spot that is spatially independently arranged.

3. The method according to claim 1, wherein the compartment is smaller than a single spot and is selected from a plurality of areas settable within a single spot.

4. The method according to claim 1, wherein the enzyme activity is the maximum production rate (Vmax) of the enzyme product.

5. The method according to claim 4, wherein calculation of the value of the maximum production rate includes a step of fitting with the Michaelis-Menten equation.

6. The method according to claim 5, further comprising calculating the affinity constant (Km) of the substrate for the enzyme.

7. An information processing apparatus for estimating enzyme activity using a base on which spots of immobilized substrates are formed, comprising:
a means for enabling an enzyme to act on the substrate on the spot to obtain an enzyme product;
a means for setting one or more compartments on the spot and obtaining density of the substrate in each compartment;
a means for measuring the density of the enzyme product in the each of the compartments; and
a means for estimating the enzyme activity of the enzyme based on a relationship between the density of the substrate in the each of the compartments and the density of the enzyme product in the each compartment.
